(19) United States
(12) Patent Application Publication
(10) Pub. No.: US 2014/0303743 A1
(43) Pub. Date: Oct. 9, 2014

(54) INNER ACETABULAR LINER FOR A DUAL MOBILITY FEMORAL HEAD CONSTRUCT

(71) Applicants: Sambhu Choudhury, Cincinnati, OH (US); Sean Lynch, Cincinnati, OH (US); Arturo David Sanchez, Cincinnati, OH (US); Andrew Ryan Law, Cincinnati, OH (US)

(72) Inventors: Sambhu Choudhury, Cincinnati, OH (US); Sean Lynch, Cincinnati, OH (US); Arturo David Sanchez, Cincinnati, OH (US); Andrew Ryan Law, Cincinnati, OH (US)

(21) Appl. No.: 14/216,798

(22) Filed: Mar. 17, 2014

Related U.S. Application Data
(60) Provisional application No. 61/798,742, filed on Mar. 15, 2013.
(60) Provisional application No. 61/813,836, filed on Apr. 19, 2013.

(51) Int. Cl. A61F 2/34

(52) U.S. Cl. CPC A61F 2/34; A61F 2002/348
USPC 623/22.24

(57) ABSTRACT

A dual mobility femoral head construct has a secure acetabular shell received within an acetabular recess formed in a pelvis. An implantable prosthetic device is implantable after a failure of an originally installed articular head insert of an acetabular cup assembly. A hemispherical liner is formed from a cast cobalt-chromium alloy for thinness and elasticity. At least three spacers are annularly displaced about an outer diameter of the hemispherical liner to define a uniform cement thickness with the secure acetabular shell. Web shaped depressions are formed circumferentially in the outer diameter of the hemispherical liner to receive cement to resist lever out forces and to secure the cement bond with the secure acetabular shell. A replacement articular head insert is then received for dual mobility rotational movement in an inner diameter of the hemispherical liner. A femoral head implant is received for articulating movement in the articular head insert.

### CROSS-REFERENCE TO RELATED APPLICATION

This application claims priority or the benefit of U.S. Provisional Application 61/798,742 filed Mar. 15, 2014 and of U.S. Provisional Application 61/813,836 filed Apr. 19, 2013, the contents of which are fully incorporated herein by reference.

### BACKGROUND OF THE INVENTION

1. Field of the Invention

The field of art disclosed herein pertains to prosthetic medical devices, and more particularly to cementable cast CoCr metal liners in conjunction with dual mobility femoral head constructs. In some embodiments, the dual mobility femoral head constructs are metal or ceramic femoral and the cementable liner is constructed of ceramic or polyethelene materials.

2. Description of the Related Art

Total hip replacement surgery is commonly performed to alleviate pain and loss of function in injured and diseased hip joints. During this surgery, the articulating surfaces of the hip joint are replaced with prosthetic bearing components. The replacement components generally include a femoral component having a convex bearing surface and an acetabular cup component having a mating concave bearing surface.

Modular femoral and acetabular components have become popular because they allow the surgeon to assemble components in a variety of configurations at the time of surgery to meet specific patient needs relative to size and geometry. For example, modular femoral components generally include separate stem and head components that can be assembled in a variety of configurations of surface finish, stem diameter, stem length, proximal stem geometry, head diameter, and neck length. Likewise, modular acetabular components generally include separate shell and liner components that can be assembled in a variety of configurations of surface finish, shell outer diameter, liner inner diameter, and constraining fit with the femoral head.

A common clinical scenario encountered by an orthopedic surgeon is a patient with a secure cementless acetabular shell and a failed articular head insert due to failed wear properties, or instability. One treatment option is to cement a new liner into the fixed shell. This is an optimal treatment option as it retains the existing acetabular shell component without compromising existing acetabular bone stock. Unfortunately, stability is dictated by the market's current liner options, and inner diameter head options. In order to optimize stability, the outer diameter liner, with the largest inner diameter head acceptance would be cemented into the existing acetabular component. A necessary cement mantle of 0.5 mm is needed to ensure stability of the liner into the acetabular component.

### SUMMARY OF THE INVENTION

In one aspect, the present disclosure provides an implantable prosthetic device having an inner hemispherical liner formed from a cast cobalt-chromium alloy and having an outer diameter sized for attachment inside a secure acetabular shell implanted into an acetabular recess in a pelvis. At least three spacers are annularly displaced about an outer diameter of the inner hemispherical liner to define a uniform cement thickness with the secure acetabular shell. Web shaped depressions are formed circumferentially and longitudinally in the outer diameter of the inner hemispherical liner to receive cement.

In another aspect, the present disclosure provides an implantable prosthetic assembly having a secure acetabular shell received within an acetabular recess formed in a pelvis. An implantable prosthetic device has an inner hemispherical liner formed from a cast cobalt-chromium alloy and sized for attachment inside the secure acetabular shell. At least three spacers annularly are displaced about an outer diameter of the inner hemispherical liner to define a uniform cement thickness with the secure acetabular shell. Web shaped depressions are formed circumferentially and longitudinally in the outer diameter of the inner hemispherical liner to receive cement. An articular head insert is received for rotational movement in an inner diameter of the inner hemispherical liner. A femoral head implant is received for articulating movement in the articular head insert.

These and other features are explained more fully in the embodiments illustrated below. It should be understood that in general the features of one embodiment also may be used in combination with features of another embodiment and that the embodiments are not intended to limit the scope of the invention.

### BRIEF DESCRIPTION OF THE DRAWINGS

The various exemplary embodiments of the present invention, which will become more apparent as the description proceeds, are described in the following detailed description in conjunction with the accompanying drawings, in which:

FIG. 1 depicts a diagram of an isometric, exploded view of a dual mobility femoral head construct according to one embodiment.

FIG. 2 depicts a front view in vertical cross section of the dual mobility femoral head construct implanted into a pelvis and femur.

FIG. 3 depicts an isometric, top view of an inner acetabular shell for the dual mobility femoral head construct of FIG. 1 according to one version.

FIG. 4 depicts a top view of the inner acetabular shell of FIG. 3.

FIG. 5 depicts a side view of the inner acetabular shell of FIG. 3.

FIG. 6 depicts a side cross-section view of the inner acetabular shell of FIG. 5 taken along lines A-A.

FIG. 7 depicts a detail view within circle B of the inner acetabular shell of FIG. 6.

### DETAILED DESCRIPTION

The present invention relates to a prosthetic acetabular liner for replacing the natural bearing surface of the hip acetabulum. A standard total hip prosthesis comprises two parts constituting a ball-and-socket joint, namely a first part intended to be implanted in the pelvis of a patient, and a second part intended to be implanted in the femur.

The first part of the prosthesis generally has a stem which is intended to engage in the medullary canal of the femur, and of which the proximal end is connected by a neck to a spherical head, or ball, intended to engage in the female part, or socket, of the joint.

The second part of the prosthesis, which has to be implanted in the pelvis, and which will be designated generally by the word acetabulum, normally comprises a hemispherical insertion cup, which is placed in a prepared cotyloid cavity of the pelvic bone, and in which is placed an articular insert, which is designed to receive the spherical head. The insertion cup is commonly made of metal. The articular insert is made of a material with a low coefficient of friction, such as polyethylene or a ceramic.

In one embodiment of the present disclosure provides an implantable prosthetic device having a truncated hemispherical liner (for example, to extend around an arc of more than 130, 135, 140, 145, 150, 155, 160, 165, 170, 175 and 180 degrees; in one embodiment, about 165 degrees) formed from a cast cobalt-chromium alloy and having an outer diameter sized for attachment inside a secure acetabular shell implanted into acetabular components recessed in a pelvis. In one embodiment, at least three spacers are annularly displaced about an outer diameter of the liner to define a uniform cement thickness within the secure acetabular shell. Web shaped depressions are formed circumferentially and longitudinally in the outer diameter of the hemispherical liner to receive cement. This webbing is designed to resist both torsional and lever-out forces of hip kinematics.

In another aspect, the present disclosure provides the acceptance of a dual mobility femoral head construct being received into the liner, within an acetabular component recessed in a pelvis. An implantable prosthetic device has a truncated hemispherical liner formed from a cast cobalt-chromium alloy and sized for cement bonding inside the secure acetabular shell. At least three spacers annularly are displaced about an outer diameter of the truncated hemispherical liner to define a uniform cement thickness within the secure acetabular shell. Web shaped depressions are formed circumferentially and longitudinally in the outer diameter of the hemispherical liner to receive cement. This webbing is designed to resist both torsional and lever-out forces of hip kinematics.

The dual mobility head construct articulates within the inner diameter of the truncated hemispherical liner. The construct is comprised of a modular femoral head that is fixed onto a femoral prosthetic neck, which then articulates into a larger diameter modular polyethylene head. This construct optimizes wear at the modular femoral head/polyethylene interface, and optimizes stability at the polyethylene/cast CoCr liner interface. Optimizing the largest polyethylene head diameter optimizes hip stability.

Turning now to the drawings, wherein like reference numerals refer to like components throughout the several views. The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well-known structures and components are shown in block diagram form in order to avoid obscuring such concepts.

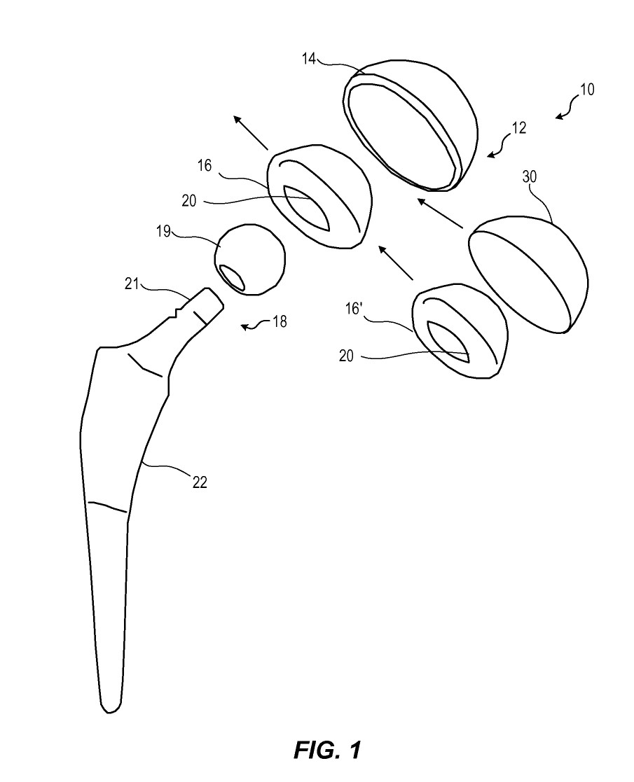

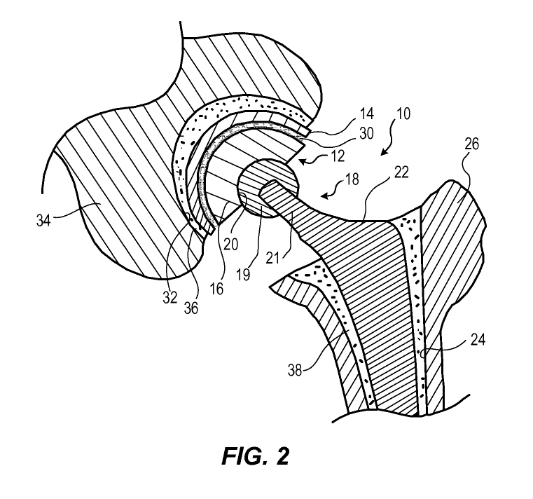

FIGS. 1-2 depict a prosthetic hip assembly, in particular a dual mobility femoral head construct 10, for replacing a human hip joint. An acetabular liner assembly 12 as originally implanted includes a secure acetabular shell 14 of a porous metal that fuses with the pelvic bone and an articular head insert 16 received for rotation therein. A femoral prosthesis 18 that is spherically shaped is received for articulating movement within a hemispherical recess 20 in the articular head insert 16. The femoral prosthesis 18 replaces the natural femoral head. The femoral prosthesis 18 includes an articulating head component 19 is assembled to distal canted end 21 of a femoral stem component 22 that is seated in a prepared intramedullary space 24 of a femur 26.

With particular reference to FIG. 1, the original articular head insert 16 that is failed is removed and replaced with an inner acetabular liner 30 sized for the inner diameter (ID) of the secure acetabular shell 14. The latter includes features discussed below to facilitate a long-lasting cement attachment to the secure acetabular shell 14. The inner acetabular liner 30 receives for movement an appropriately sized articular head insert 16′. The original acetabular shell 14, such as composed of Titanium or Tantalum, has typically not failed and is left in place. The replacement insert 16′ is generally made of biocompatible polymer such as polyethylene, polyether ether ketone (PEEK), polyaryletherketone (“PAEK”), an ultra-high molecular weight polyethylene polymer material (“UHMWPE”) such as an antioxidant stabilized UHMWPE or another equivalent plastic material. In other exemplary embodiments, the biocompatible polymer may be a polyolefin, polyester, polyimide, polyamide, polyacrylate, and/or other suitable polymers.

With continued reference to FIGS. 1-2, the acetabular liner assembly 12 lines an acetabulum 32 on the pelvic side of the hip joint. The acetabular liner assembly 12 is pressed into the prepared acetabulum 32 of a pelvis 34. The secure acetabular shell 14 may abut the bone or a layer of bone cement 36 may be positioned within the acetabulum 32 and the secure acetabular shell 14 to lock the acetabular liner assembly 12 in place. The femoral stem component 22 may abut bone of the femur 26 or a layer of bone cement 38 may be positioned between the bone and the femoral stem component 22 of the acetabular liner assembly.

The articulating head component 19 may be permanently affixed to the femoral stem component 22 or it may be a modular piece fit on the femoral stem component 22 at the time of surgery. After the acetabular liner assembly 12 and femoral stem component 22 has been implanted, the articulating head component 19 is inserted into the concave bearing surface 40 of the articular insert 16 to restore normal hip joint function. In one embodiment, the articulating head 19 is produced from a biocompatible metal, e.g. a titanium alloy, a cobalt-chromium alloy, or a stainless steel alloy and may be coated by a hard, low friction coating such as amorphous diamond-like coating (ADLC). Alternatively, the head may be produced from carbon, e.g. a pyrolytic carbon, with essentially the same surface characteristics as ADLC.

By way of example but not limitation, the articulating head 19 may comprise of cobalt chrome, titanium, titanium alloys, tantalum, tantalum alloys or other metals and/or metal alloys consistent with the present invention. Among other things, the articulating head 19 is preferably a material that is highly biocompatible. By way of further example but not limitation the articulating head 19 may comprise CoCrMo, cobalt based alloys, stainless steels, zirconium based alloys or other metals and/or metal alloys consistent with the present invention. In one embodiment, the articulating head 19 is preferably a material which has relatively high hardness and which is scratch resistant. For the purposes of the present invention, the term bimetal may be defined as a composite of two metals, where each of the metals has a different primary constituent. The bimetal construct can be formed from two different commercially pure metals, from two alloys of different base metals, or a combination thereof.

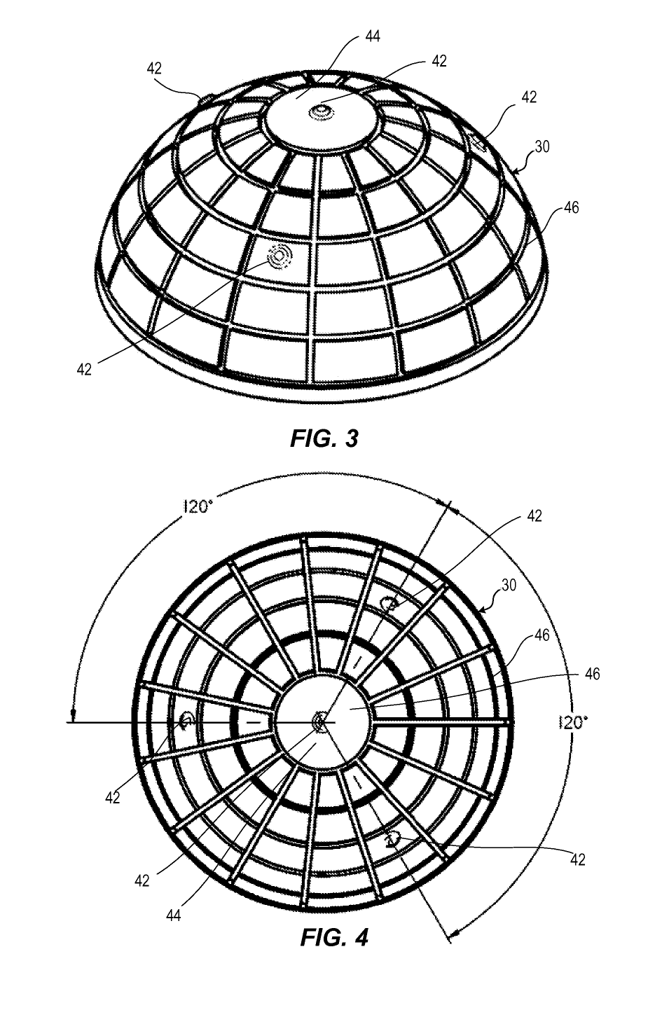

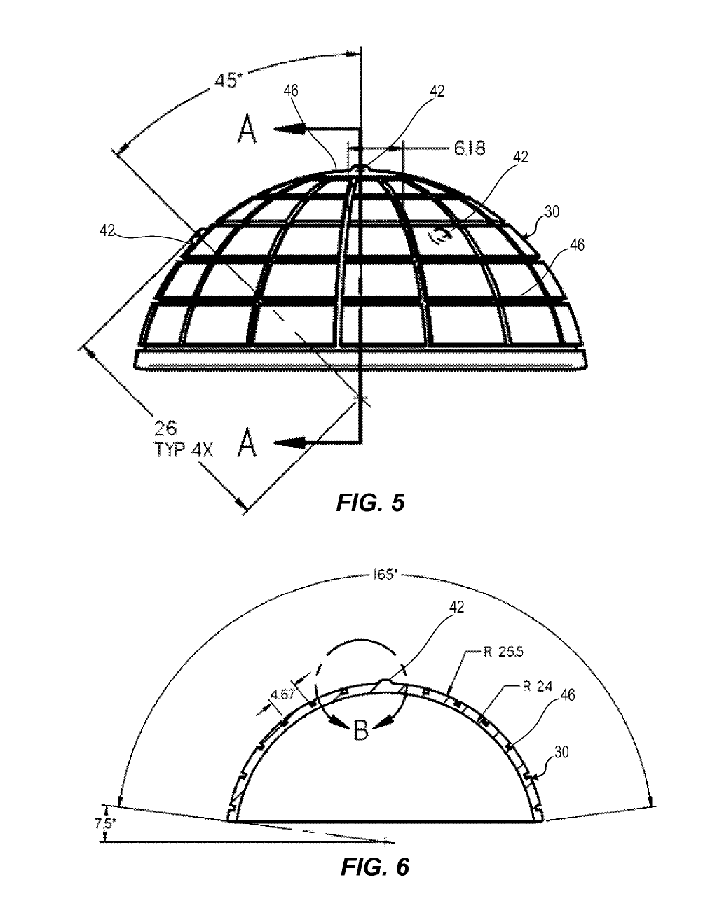

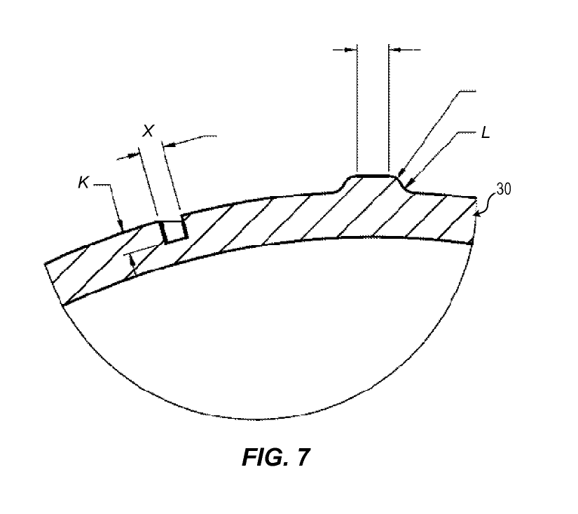

With reference to FIGS. 3-7, the new inner acetabular liner 30 is depicted in various orientations and details to depict features for facilitating fixation of liner 30 to the secure acetabular liner 14. The connection between liner 30 to the secure acetabular liner 14 could be a direct connection with the contacting surface, or an indirect connection with the contacting surface. In the embodiments where the connection is an indirect connection, a material could be positioned between the new liner 30 and the secure acetabular shell 14. The material could be a material selected from a group consisting of: adhesive materials, elastic materials and bone cement. The material could be at least one of: bone cement, an at least partly elastic material, glue, adhesive, antibiotic, biocompatible plastic material, biocompatible ceramics or biocompatible metal.

However it is also conceivable that the connection is assisted or replaced with at least one screw, at least one pin, at least one portion of at least one of the parts adapted to be introduced into the other part, the parts being adapted to be sliding into the other part, form fitting, welding, adhesive, pin, wire, a ball mounted into portions of the parts, a male portion of one part mounted into a female portion of the other part, a key introduced into a lock portion of parts, band, or other mechanical connecting members. The new inner acetabular liner 30 can have surface features such as textures, grooves, knurling, etc.

In some embodiments, the new inner acetabular liner 30 can be held in place to the secure acetabular shell 14 by a friction fit. In some embodiments, a retention material, such as adhesives can be used to hold the new inner acetabular liner 30 in place to the secure acetabular shell 14. In other embodiments, the new inner acetabular liner 30 can have a mating feature that couples with a complementary mating feature on the secure acetabular liner 14, such as hooks, splines, tabs, channels and grooves, or any other mating features as would be known in the art.

In one embodiment, the liner 30 is attached using an adhesive. In one embodiment, the adhesive is a biocompatible adhesive such polymethylmethacrylate (PMMA), POROCOAT or other non-resorbable cement. The cement is generally in liquid or paste form but may be a powder or solid to which a liquid component is added.

In one embodiment, the new inner acetabular liner 30 is a cementable liner comprising metal, ceramic, polymer or combinations thereof. In one embodiment, the new inner acetabular liner 30 is formed from a medical-grade metal such as stainless steel, cobalt chromium, or titanium, although other metals or alloys may be used. As an example, the liner 30 may be a ceramic liner, such as an alumina ceramic liner. Further examples include a diamond liner, a liner made of a polycrystalline diamond composite material, a liner made from oxidized Zirconium, or a liner made from polyethylene, including cross-linked polyethylene. Moreover, in some embodiments, rigid polymers such as polyetheretherketone (PEEK) may also be used. In one embodiment, titanium alloy or cobalt chromium alloy may be used. However, if the components, and in particular the new inner acetabular liner 30, are more modular in nature, then different materials can be considered to improve workability, cost-savings, implantation, and ultimately use by the patient once implanted. In one embodiment, the new inner acetabular liner 30 is formed from polyethylene. Further, an interior or an exterior of the liners may be coated with various types of coatings. For example, these surfaces may have a metal, plastic, diamond, zeolite or composite coating.

In one embodiment, the inner acetabular liner 30 is formed from cast a cobalt-chromium (CoCr) alloy. For example, the inner acetabular liner 30 may have a highly polished Inner Diameter (ID) starting at 36 mm ID and 40 mm outer diameter (OD). Using cast CoCr alloy allows for a very thin liner, optimizing the ID of the liner, the size of the dual mobility head, and stability of the hip. The polished ID optimizes wear characteristics between the inner acetabular liner 30 and the articular head insert 16. The cast technique also allows for a higher elasticity than a forged CoCr liner, which may optimize liner/cement interface stability.

In another embodiment, with particular reference to FIGS. 3-5, polymethyl methacrylate (PMMA) spacers 42 marked at the apex of the OD and on each side of the inner acetabular liner 30. For example, three PMMA spacers 42 may be annularly spaced 120° a part at the mid-point of radius of curvature in addition to the apex. It should be appreciated that PMMA material is illustrative and that other materials may be used. These PMMA spacers 42 allow a uniform cement mantle of at least 0.5 mm between the secure acetabular shell 14 and apex of the CoCr OD of the inner acetabular liner 30 and 1.25 mm of cement interdigitation within the longitudinal and circumferential and longitudinally “depressed” spider webbing 46. Uniformity of cement mantle is important for mechanical stability.

In another embodiment, the spider webbing 46 has a 0.75 mm depressed design with 0.5 mm pole to optimize lever out and torsional forces by cement/liner interdigitation and stability.

In one embodiment, the inner acetabular liner 30 has an inner surface and an outer surface with the inner surface sized and shaped to compliment the outer portion of the articular head insert 16, and the outer surface is sized and shaped to compliment the inner wall of a secure acetabular shell 14. The thickness of the inner acetabular liner 30 may vary from about one-half millimeter to about 30 millimeters. In another embodiment, the thickness of the inner acetabular liner 30 may vary from about one-half millimeter to about ten millimeters.

In some embodiments, the inner acetabular liner 30 may fit differently sized shells. As an example only, the liner may fit inner diameter shells having an inner diameter from about 25 to about 75 mm. In another embodiment, the liner may fit inner diameter shells having an inner diameter from about 35 to about 65 mm.

In another embodiment, with particular reference to FIG. 2 and, a truncated 165° design stops short of a true 180° hemispherical design to avoids neck impingement on the inner acetabular liner 30. Older designed stem tended to have larger necks creating earlier impingement during Range of Motion (ROM). When using this product in a revision setting avoids early impingement and potential dislocation of the hip.

According to a another embodiment, there is provided a kit of parts for a prosthesis, the kit of parts comprising one or more inner acetabular liners 30 and one or more corresponding articular head insert adapted to fit the one or more inner acetabular liners 30. The kit of parts may further comprise a cement and an insertion tool. As used herein, the term “kit” or “system” is used to denote a collection of readily available components (i.e., acetabular cups, bearings and/or femoral hip prostheses) available for a surgeons easy selection. The collection of readily available components can be grouped or arranged in any manner that provides the surgeon with comprehensive access and ease of identification. In this way, each of the components may be prepackaged individually in sterile containers while still being offered as a collective kit or set of components.

In the known devices, a distinction can be made between single-mobility head/liner constructs, dual-mobility head/liner constructs, within or excluding modular acetabular systems. In the single-mobility head/liner constructs, the polyethylene or ceramic insert is fixed in an insertion cup and has a coaxial and substantially hemispherical articular cavity permitting the engagement and pivoting of the spherical head of the first part of the prosthesis. The rotation movements of the joint then take place between the spherical head of the first part of the prosthesis and the articular cavity of the articular insert fixated within a modular acetabular shell system. In a dual-mobility head/liner construct, the articular insert is itself mounted rotatably in the insertion cup, thereby providing a first sliding surface between the insertion cup and the articular insert, and a second sliding surface between the articular insert and the spherical head. This head articulates within a non-modular acetabular shell, or a modular acetabular shell with a liner. In the acetabulums with a non-modular acetabular system, the articular insert has a spherical outer surface so as to be rotatably mounted directly in the cotyloid cavity of the pelvis of a patient. Alternatively, a modular acetabular system has a spherical outers surface so as to be rotatably mounted directly in the cotyloid cavity of the pelvis of a patient, and receive a fixate modular articulating surface comprised of numerous bearing surface material options.

All publications, patents and patent applications cited herein, whether supra or infra, are hereby incorporated by reference in their entirety to the same extent as if each individual publication, patent or patent application was specifically and individually indicated as incorporated by reference. It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein, will only be incorporated to the extent that no conflict arises between that incorporated material and the existing disclosure material.

It must be noted that, as used in this specification and the appended claims, the singular forms “a,” “an” and “the” include plural referents unless the content clearly dictates otherwise. Thus, for example, reference to a “colorant agent” includes two or more such agents.

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

As will be appreciated by one having ordinary skill in the art, the methods and compositions of the invention substantially reduce or eliminate the disadvantages and drawbacks associated with prior art methods and compositions.

It should be noted that, when employed in the present disclosure, the terms “comprises,” “comprising,” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

While it is apparent that the illustrative embodiments of the invention herein disclosed fulfill the objectives stated above, it will be appreciated that numerous modifications and other embodiments may be devised by one of ordinary skill in the art. Accordingly, it will be understood that the appended claims are intended to cover all such modifications and embodiments, which come within the spirit and scope of the present invention.

What is claimed is:

1. An implantable prosthetic device, comprising:
a hemispherical liner formed from a cast cobalt-chromium alloy and having an outer diameter sized for attachment inside a secure acetabular shell implanted into a acetabular recess in a pelvis;
at least three spacers annularly displaced about an outer diameter of the hemispherical liner to define a uniform cement thickness with the secure acetabular shell; and
web shaped depressions formed circumferentially and longitudinally in the outer diameter of the hemispherical liner to receive cement.

2. The implantable prosthetic device of claim 1 wherein the liner further comprises materials selected from the group consisting of polyethelene, ceramic and diamond.

3. The implantable prosthetic device of claim 1, wherein the at least three spacers comprise polymethyl methacrylate (PMMA) and wherein the at least three spacers comprise a first spacer attached to an apex of the hemispherical liner and at least three spacers annularly spaced at a midpoint of a radius of curvature of the outer diameter.

4. The implantable prosthetic device of claim 1, wherein the at least three spacers extend 0.5 mm from the outer diameter.

5. The implantable prosthetic device of claim 1, wherein the web shaped depressions are 0.75 mm deep.

6. The implantable prosthetic device of claim 1, wherein the inner hemispherical liner comprises a truncated radius of curvature limited to 165° with respect to a center of articulating movement of a femoral head received in an articular head insert received in turn for dual mobility by the hemispherical liner.

7. An implantable prosthetic assembly, comprising:
a secure acetabular shell received within an acetabular recess formed in a pelvis;
an implantable prosthetic device, comprising:
a hemispherical liner formed from a cast cobalt-chromium alloy,
at least three spacers annularly displaced about an outer diameter of the hemispherical liner to define a uniform cement thickness with the secure acetabular shell, and
web shaped depressions formed circumferentially and longitudinally in the outer diameter of the hemispherical liner to receive cement;
an articular head insert received for rotational movement in an inner diameter of the hemispherical liner; and
a femoral head implant received for articulating movement in the articular head insert.

8. The implantable prosthetic assembly of claim 7, wherein the at least three spacers comprise polymethyl methacrylate (PMMA).

9. The implantable prosthetic assembly of claim 7, wherein the at least three spacers comprise a first spacer attached to an apex of the hemispherical liner and at least three spacers annularly spaced at a midpoint of a radius of curvature of the outer diameter.

10. The implantable prosthetic assembly of claim 7, wherein the at least three spacers extend 0.5 mm from the outer diameter.

11. The implantable prosthetic assembly of claim 7, wherein the web shaped depressions are 0.75 mm deep.

12. The implantable prosthetic assembly of claim 7, wherein a radius of curvature of the hemispherical liner is limited to 165° with respect to a center of articulating movement of a femoral head received in an articular head insert received in turn for dual mobility by the hemispherical liner.
